(19) United States
(12) Patent Application Publication
(10) Pub. No.: US 2021/0369399 A1
(43) Pub. Date: Dec. 2, 2021

(54) NEGATIVE PRESSURE AEROSOL CONTAINMENT UNIT

(71) Applicants: Tony KIM, Houston, TX (US); Mario CALDERARA, The Woodlands, TX (US)

(72) Inventors: Tony KIM, Houston, TX (US); Mario CALDERARA, The Woodlands, TX (US)

(21) Appl. No.: 17/334,183

(22) Filed: May 28, 2021

Related U.S. Application Data
(60) Provisional application No. 63/031,369, filed on May 28, 2020.

(51) Int. Cl. A61B 90/40; A61B 46/00

(52) U.S. Cl. CPC A61B 90/40; A61B 2090/401; A61B 46/00

(57) ABSTRACT

A device is disclosed for use by clinicians in medical and dental procedures to contain aerosols generated by patients with known or suspected respiratory pathogen (e.g., a virus such as COVID-19). The device of the invention (a negative pressure aerosol containment unit) may be in the form of a box with an opening at the bottom, the box configured to receive an attached HEPA filter and HVAC unit that creates a localized negative pressure area within the box. The device also may be in a non-rectilinear configuration.

### CROSS-REFERENCE TO RELATED APPLICATIONS

This application claims the benefit of priority to U.S. Provisional Application No. 63/031,369, filed May 28, 2020 in the U.S. Patent and Trademark Office. All disclosures of the document named above are incorporated herein by reference.

### FIELD OF INVENTION

This invention relates generally to a device used in medical and dental procedures and more particularly to a new device and method used by medical and dental clinicians to contain aerosols generated by patients with respiratory infections, including infections from a known or suspected pathogen (e.g., COVID-19 virus).

### BACKGROUND

A novel coronavirus was identified in late 2019 as the cause of a cluster of pneumonia cases in Wuhan, China. It has since rapidly spread resulting in a pandemic designated as COVID-19. COVID-19 is part of the same family of coronaviruses that created Severe Acute Respiratory Syndrome (SARS-CoV-1) and Middle East Respiratory Syndrome (MERS-CoV). COVID-19, also designated SARS-CoV-2, spreads primarily from person to person via droplet spread from infected individuals when they aerosolize the pathogen. COVID-19 and other types of respiratory pathogens can infect healthy individuals through spread from an infected individual via droplet or airborne transmission. The major morbidity and mortality from COVID-19 is largely due to acute viral pneumonitis that evolves to acute respiratory distress syndrome (ARDS) and multi-system organ failure.

Aerosol Generating Procedures (AGPs) are any procedure that can generate infectious aerosols. Respiratory aerosols can vary in size from 0.6 μm to 1000 μm in diameter. In about one percent of patients with COVID-19 require endotracheal intubation and mechanical ventilation to help keep their oxygen levels stable. Endotracheal intubation is one type of AGP. In order to provide this support, clinicians and health care providers (e.g., anesthesiologists, nurse anesthetists, and emergency or critical care physicians) must place into the windpipe of a patient a breathing tube, or endotracheal tube, so the patient can receive higher levels of oxygen and breathing support on a ventilator. Placing a breathing tube in patients with COVID-19 can pose a high risk to health care workers, as it may expose them to infectious aerosols from the patient's airways. Participating in intubation procedures has been linked to clinicians developing COVID-19.

Other Aerosol Generating Procedures such as bronchoscopy, upper endoscopy, and airway surgery can also increase the risk of droplet and airborne pathogen exposure to healthcare workers. Data suggest front-line healthcare workers have a higher incidence of COVID-19 than the general public due to these types of exposures.

The Center for Disease Control recommends that infected individuals requiring Aerosol Generating Procedures be placed in Airborne Infection Isolation Rooms (AIIRs) to reduce the likelihood of exposure to pathogens. Airborne Infection Isolation Rooms have a negative atmospheric pressure relative to the surrounding areas causing airflow into a room. This airflow prevents airborne pathogens from moving out of an AIIRs due to the minimum of 6-12 air exchanges per hour.

Most hospitals only have a small number of AIIRs designated for infectious respiratory patients and therefore do not have sufficient number of AIIRs to handle all of the Aerosol Generating Procedures that need to be performed on infected patients during a significant epidemic or pandemic. Additionally, almost all hospitals have many more positive pressure rooms, such as surgical operating rooms, relative to AIIRs. Positive pressure rooms are very good for preventing surgical patients from being exposed to possible contaminants in the air due to high air circulation and the ambient atmospheric pressure being higher than the surrounding area. For a droplet or airborne pathogen, a positive pressure room increases the possibility of exposure to personnel outside the room since airborne contaminants can leak out of the room from the positive pressure. There is a significant risk of front-line healthcare workers contracting COVID-19 and other respiratory pathogens from infected patients in hospital settings given the limited number of AIIRs rooms.

The risk of contracting COVID-19 or other respiratory pathogens applies in dental settings as well as in medical settings. Many dental procedures, including but not limited to teeth cleaning and root canals, generate aerosolized droplets or airborne pathogens, thus putting dental workers as well as other patients in a dental office or clinic at risk.

In view of the above, there is a need for a negative pressure aerosol containment unit to mitigate aerosol generation during procedures performed on patients with a known or suspected contagious respiratory pathogen, including but not limited to COVID-19. Thus, the principal object of the invention is to provide such a medical device and to develop methods of using the same.

### SUMMARY

Broadly speaking, the objects of the invention are realized, according to one aspect of the invention, through a device that provides a localized negative pressure environment in the space around a patient with a known or suspected contagious respiratory pathogen and allows a clinician (medical or dental) to more safely perform necessary medical procedures such as intubation, extubation, bronchoscopy, endoscopy, other airway procedures and dental procedures (e.g., teeth cleaning, fillings, root canals, tooth extractions).

According to one aspect of the invention, the device (a negative pressure aerosol containment unit (NP-ACU)), includes a box with an opening at the bottom, the box configured to receive an attached HEPA filter and HVAC unit that creates a localized negative pressure area within the area in and around the device. The box can be fitted with different access portals to allow a healthcare or dental worker to perform aerosol generating procedures on a patient infected or suspected of infection with a respiratory pathogen. By creating a negative pressure area that filters air from the immediate vicinity where infectious aerosols are being generated, the device of the invention reduces the likelihood of healthcare or dental worker exposure to an infectious pathogen.

One aspect of the invention embraces a device for use in medical or dental procedures to contain infectious aerosols generated by a patient. The device is transparent, has an opening at the bottom adapted to fit over the patient, and is configured to receive an attached HEPA filter and HVAC unit that creates a localized negative pressure area within the device. The device may be configured to receive an attached HEPA filter and HVAC unit at more than one location, e.g., at either one or both sides of a unit with a rectilinear configuration. Further, the device may be mobile and portable, such that it may be moved into a location as needed (e.g., an operating room or emergency room). And the height of the device may be adjustable.

According to one aspect of the invention, the device is in the form of a box having an open bottom. The box may be defined by a front panel, a back panel, two side panels, a top panel and the open bottom. In one embodiment of the invention, the side panels of the box are made of a clear plastic (e.g., plexiglass) or non-porous material.

According to one aspect of the invention, the device is positioned on a mobile or portable base.

According to one aspect of the invention, the patient has a known or suspected virus. The virus may be, for example, COVID-19.

According to one aspect of the invention, the device is used to contain aerosols generated by a patient with a known or suspected contagious respiratory pathogen during intubation, extubation, bronchoscopy, upper endoscopy, and other airway medical procedures.

According to an aspect of the invention, the device is used to contain aerosols generated by a patient with a known or suspected contagious respiratory pathogen in a dental setting for procedures including but not limited to teeth cleaning, fillings, root canals, and extractions.

In one embodiment of the invention, the HEPA filter has a filtration efficiency in the range of 85% to 99.999995% of particles 0.3 μm in diameter. In another embodiment of the invention, the HEPA filter has a filtration efficiency in the range of 99.0% to 99.999995% of particles 0.3 μm in diameter. In another embodiment of the invention, the HEPA filter has a filtration efficiency in the range of at least 99% of particles 0.3 μm in diameter. In another embodiment of the invention, the HEPA filter has a filtration efficiency in the range of at least 99.999995% of particles 0.3 μm in diameter.

In one embodiment of the invention, the HVAC unit pulls air at over 200 cubic ft./minute. In one embodiment of the invention, the HVAC unit pulls air at over 250 cubic ft./minute. In another embodiment of the invention, the HVAC unit pulls air in a range of 250 cubic ft./minute-600 cubic ft./minute.

In one embodiment of the invention, the front panel of the device is adapted for two openings through which a clinician can engage with the patient using his hands.

In one embodiment of the invention, at least one side panel has an adapted opening through which a clinician can engage the patients using his hands.

In another embodiment of the invention, the back panel is adapted to fit over the patient's torso or abdomen.

According to an embodiment of the invention, one side panel is configured to be fitted with the HEPA filter. The other side panel may have a large opening configured to be covered with a drape which will allow access by an assistant clinician.

According to another embodiment of the invention, the top panel of the box is configured to be fitted with the HEPA filter.

### BRIEF DESCRIPTION OF THE DRAWINGS

The invention is more fully described by reference to the following detailed description and the accompanying drawings wherein:

FIGS. 1A and 1B show an embodiment of the aerosol containment unit according to the invention with a rolling, mobile base and various slots for HVAC connection and operator portals. FIG. 1A provides a photograph of this embodiment, and FIG. 1B provides a drawing of this embodiment.

FIGS. 2A and 2B show an embodiment of the aerosol containment unit according to the invention with an emphasis on the connection location for the HEPA Filter HVAC duct. FIG. 2A provides a photograph of this embodiment, and FIG. 2B provides a drawing of this embodiment.

FIGS. 3A and 3B illustrate an embodiment of the aerosol containment unit according to the invention showing the hand portals for the operator to be able to access the patient's head to perform an aerosol generating procedure such as intubation or extubation. FIG. 3A provides a photograph of this embodiment, and FIG. 3B provides a drawing of this embodiment.

FIGS. 4A and 4B illustrate an embodiment of the aerosol containment unit according to the invention showing the side portal for a medical or dental assistant to be able to access the box and help the primary operator working at the head of the box. FIG. 4A provides a photograph of this embodiment, and FIG. 4B provides a drawing of this embodiment.

FIG. 5 is a photograph of a HEPA filter HVAC unit that can attach to the clear plastic side of an aerosol containment unit to create negative pressure internally within the aerosol containment unit of the invention.

FIG. 6 illustrates how a HEPA filter HVAC unit is attached to the clear plastic box to create the negative pressure aerosol containment unit according to the invention.

FIG. 7 illustrates the position of the aerosol containment unit according to the invention on a patient bed.

FIGS. 8A and 8B illustrate how an embodiment of the aerosol containment unit according to the invention would be used on a patient in the operating room. FIG. 8A provides a photograph showing this aspect of the invention, while FIG. 8B provides a drawing showing this aspect of the invention.

FIGS. 9A-9E show different views of an embodiment of the invention. FIG. 9A is an isometric view of an embodiment of the device of the invention, the aerosol containment unit in the form of a box and defined by a front panel, a back panel, two side panels, a top panel and an open bottom. FIG. 9B is a front view of the aerosol containment unit according to the invention. FIG. 9C is a side view of the aerosol containment unit according to the invention.

FIG. 9D shows a rear overhang panel on the posterior side of the aerosol containment unit according to the invention. FIG. 9E is top view of the aerosol containment unit of the invention showing the top panel.

FIG. 10 shows a non-rectilinear embodiment of the invention.

### DETAILED DESCRIPTION

According to one aspect of the invention, the device (also known as the negative pressure aerosol containing unit, or NP-ACU) is a clear plastic (e.g., plexiglass) box mounted on a rolling frame that can be raised or lowered in a manner similar to bedside tables used in other parts of a hospital. The device is used in part to serve as a negative-pressure chamber to help reduce aerosol generation during intubation and extubation for patients with known or suspected respiratory pathogens such COVID-19 (COVID+/PUI).

The device is not limited to only a hospital setting. Indeed, the device may be used in other medical or dental settings, including in a dental office or clinic. Dental professionals such as dentists or dental technicians are particularly vulnerable to exposure to aerosols generated by patients during dental procedures. Such procedures may include but are not limited teeth cleaning, fillings, extractions, and root canals. In certain cases, the patients may have a respiratory infection and the aerosols generated by the patients can spread pathogens to dental professionals and other patients in the dental setting.

The device is not limited in shape to a rectilinear configuration (e.g., a box, square or rectangular configuration), but rather may be configured in a variety of shapes (e.g., a three dimensional oval shape or a cylindrical shape) and may be adapted to fit over top a patient in a rested position (e.g., as the patient is positioned in a dental chair or an operating room table).

According to one aspect of the invention, a high-quality HEPA filter with a viral filtration efficiency in the range of 85% to 99.999995% (of particles 0.3 μm in diameter) is attached to a side panel of the box. This filter is connected to a vacuum which pulls over 250 cubic feet per minute of air (7000 liters per minute). In another embodiment, the vacuum pulls air in a range of 250 to 600 cubic ft/minute. The opposite side panel of the box will have a large opening which should be initially closed with a drape. This opening is intended for access if an assistant clinician (e.g., an airway assistant, nurse or dental technician) is required. The front of the NP-ACU has two holes for the primary clinician's (e.g., anesthesiologist's) hands, and the posterior side is open or partially open, allowing space for the patient's torso or abdomen.

According to an aspect of the invention for a non-operating room environment, the device includes a clear plastic (e.g., plexiglass) box mounted with handles on three (3) sides which allows the box to be positioned on top of a patient while they are on a bed, stretcher, or gurney. One purpose of the device is to serve as a negative-pressure chamber to help reduce aerosol generation during medical procedures (e.g., intubation and extubation) or dental procedures for patients with known or suspected respiratory pathogens such as COVID-19 (COVID+/PUI).

According to an aspect of the invention, the device is a clear plastic (e.g., plexiglass) box with different portals created for surgeons, proceduralists, and airway specialists to manage a patient having an aerosol generating procedure. One purpose of the device is to serve as a negative-pressure chamber to help reduce aerosol generation during other types of aerosol generating procedures such as bronchoscopy, upper endoscopy, tracheostomy for patients with known or suspected respiratory pathogens such COVID-19 (COVID+/PUI).

A video laryngoscope may be placed, for example, on the operator's left side, and the video scope should be brought in underneath the edge of the device (e.g., a box-shaped device) and placed near the patient's head. Likewise, in the case of a box-shaped device, the anesthesia circuit may be brought in from the right side under the edge of the box.

In an aspect of the invention with a patient who has contracted COVID-19, full COVID personal protective equipment (PPE), including droplet plus airborne precautions (N95 mask & face shield min), should still be utilized by all healthcare staff in the room on any COVID+, PUI, or high aerosolizing procedure. For safety purposes, it is worth noting that the device of the invention may not completely eliminate the risk of aerosol generation.

In the event of a difficult intubation, the assistant clinician should slit a hole in a drape on the right side of the device and provide any support as needed. The device of the invention has been tested with a Bougie catheter, and it has operated successfully. A Bougie catheter is much easier to use if an assistant is available to help.

In a cannot-intubate, cannot-oxygenate scenario, the device of the invention may be rapidly raised and backed up away from the patient. This will provide access to the front of the neck if a surgical airway is required.

If the patient is to be extubated in the operating room, the device of the invention may also be placed over the patient for extubation and/or recovery. This should help reduce aerosol generation if the patient coughs or vomits during the extubation procedure.

The device of the invention should be kept on during a procedure regardless of whether it is used on the patient or not. The filtration aspect of the HVAC unit will help keep the air in the procedural room cleaner.

After each procedure, the device of the invention may require a thorough wipe down and decontamination with appropriate wipes. Other means of decontaminating the device also may be used. The HEPA filter in the HVAC unit does not need to be changed or cleaned immediately after each use; it may be used again in other procedures. The exterior of the HVAC unit, however, should be wiped down after each procedure.

A thin pre-filter may be used on the inside rim of the device where the HVAC duct connects. This pre-filter should be disposed of between uses and a new pre-filter installed.

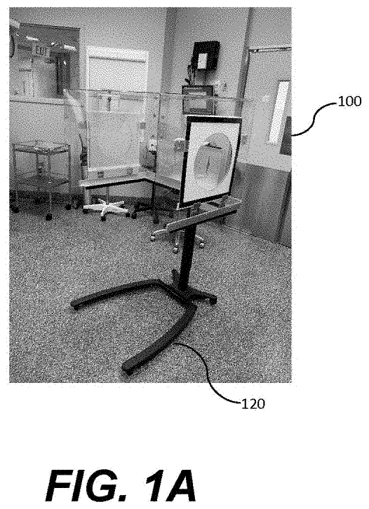

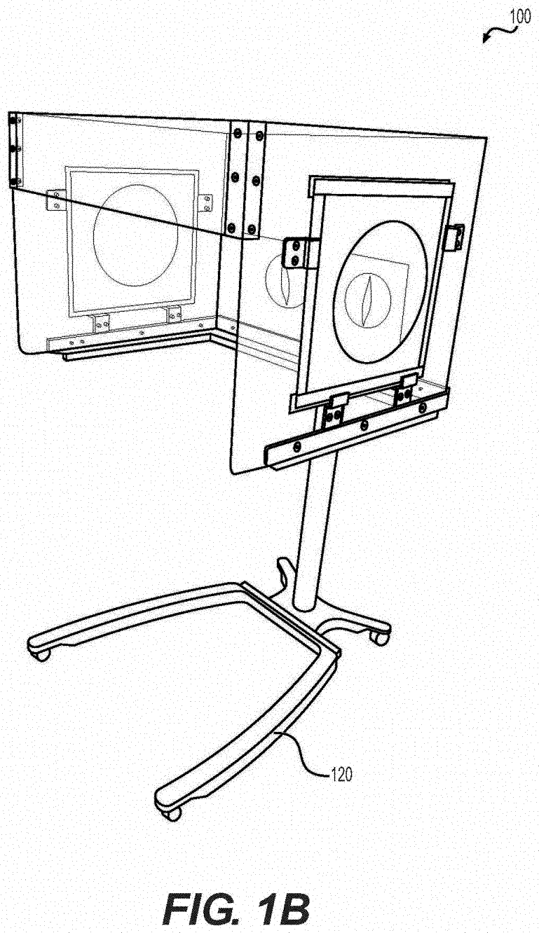

Referring more specifically to the drawings, FIGS. 1A and 1B exemplify an embodiment of the invention in which the aerosol containment unit (or device) 100 is positioned on a mobile base 120. Mobility and portability of the device of the invention is an important optional feature, as it allows hospital or dental staff to easily locate the invention in the room of greatest need. While it is not shown in these figures, the device of the invention may be raised or lowered with respect to a patient in a manner similar to bedside tables used in other parts of a hospital or medical facility. In other aspects of the invention, the device may be raised or lowered over dental chairs. Additional figures will show other features of the invention with more clarity.

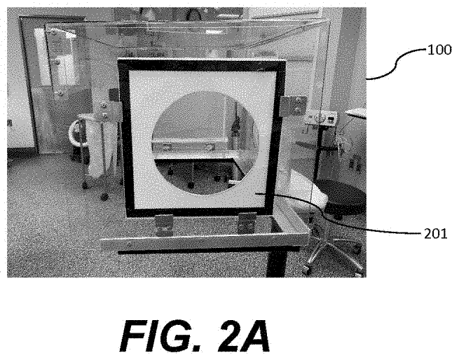

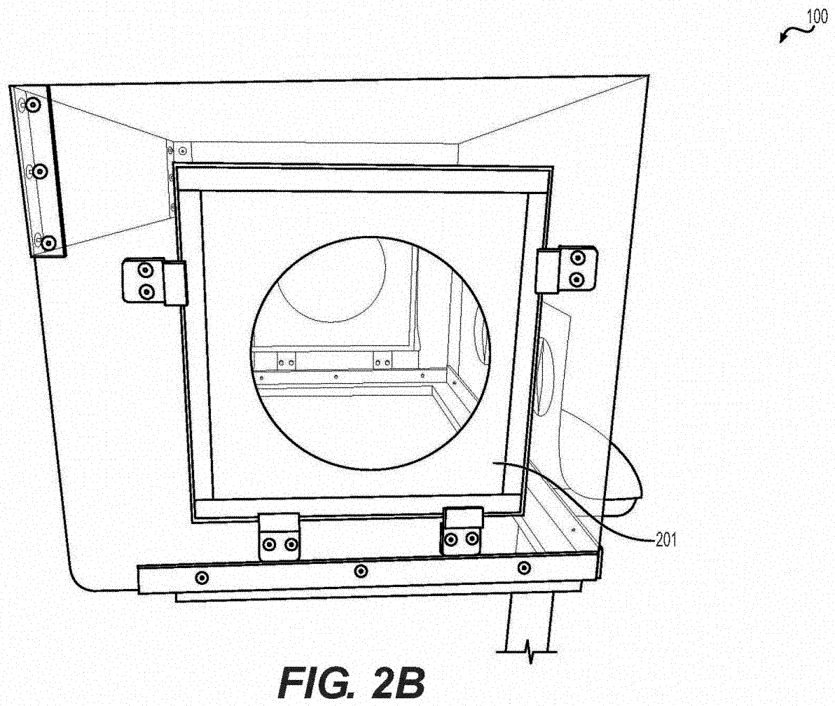

FIGS. 2A and 2B show an embodiment of the aerosol containment unit with an emphasis on the connection 201 for the HEPA Filter HVAC duct. The connection for the HEPA filter HVAC duct may be located on one or both side panels of the aerosol containment unit.

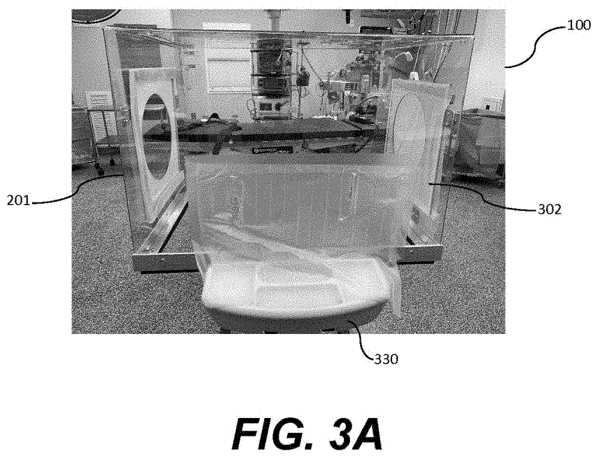

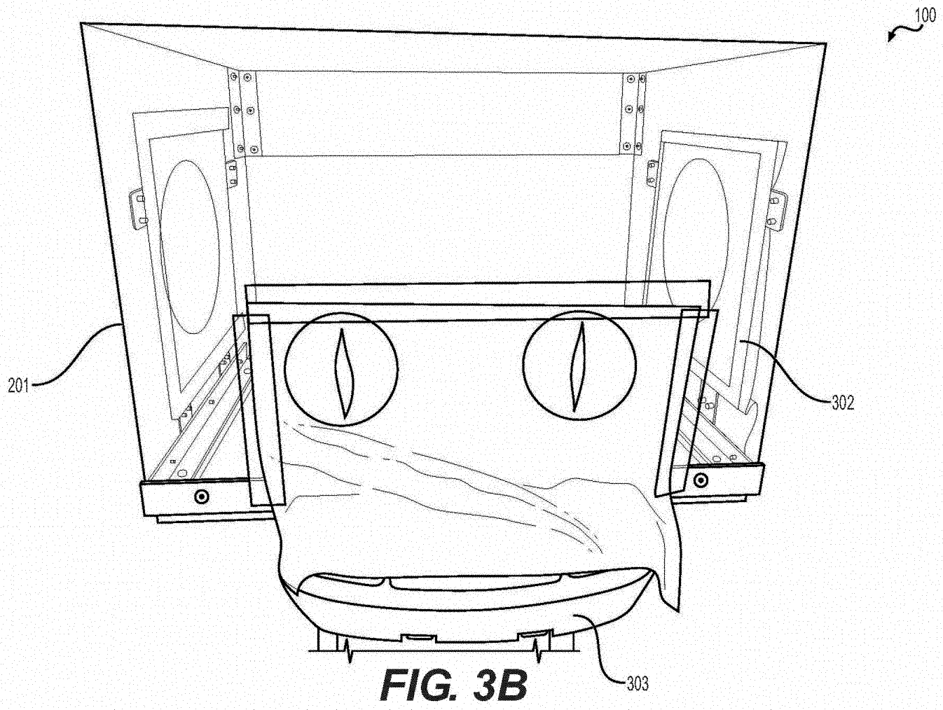

FIGS. 3A and 3B provide a front view of an embodiment of the invention showing the hand portals 302 for the operator to be able to access the patient's head to perform an aerosol generating procedure such as intubation or extubation. In these figures, the hand portals are draped with a flexible plastic cover. An optional table 303 is connected to the device in the front to allow the primary clinician to rest the tools and equipment necessary for the procedure.

The panels of the invention in this embodiment are made of clear plastic (e.g., plexiglass) to provide a clear view of the patient from the top and all four sides of the device. Other plastics as well as glass may be used in the device. It is important that the materials used in the device are clear and transparent.

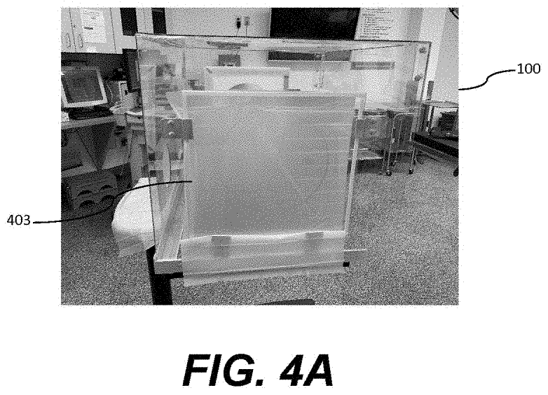

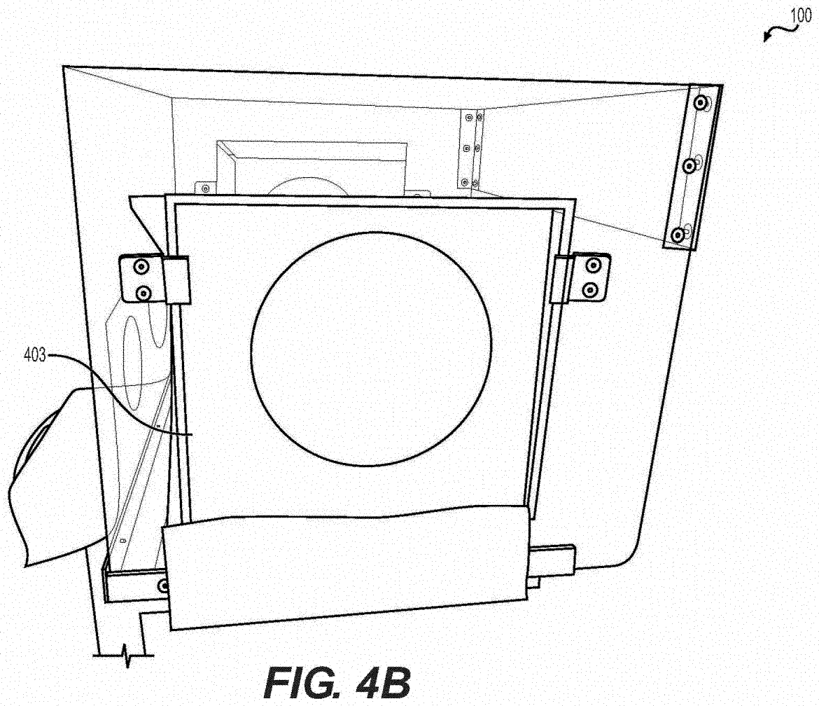

FIGS. 4A and 4B show an embodiment of the aerosol containment unit showing the side portal 403 for a medical or dental assistant to be able to access the box and help the primary operator working at the head of the box. When not in use, the aperture at the side portal should be covered with one or more drapes or other barrier (e.g., ten-ten drapes) to prevent unnecessary leaks of air into the device. The drapes may be slit to allow the medical or dental assistant to place their arms into the device while at the same time minimizing leakage of air into the low pressure environment of the device.

A medical assistant may be helpful in performing adjunct tasks, such as touching the anesthesia machine, turning on the ventilator, and helping with difficult intubations.

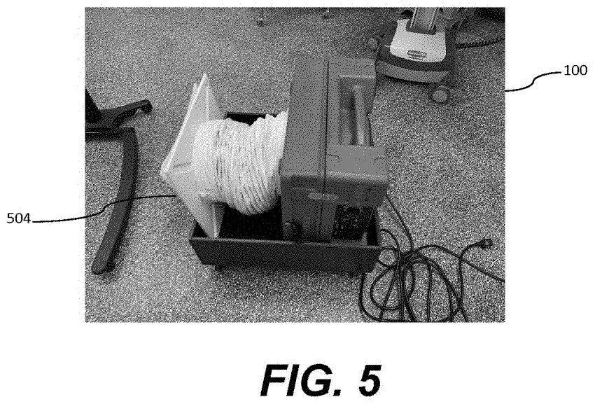

FIG. 5 is a photograph of a HEPA filter HVAC unit 504 that can attach to the clear plastic (e.g., plexiglass) side of an aerosol containment unit to create negative pressure internally within the unit. A high-quality HEPA filter with a viral filtration efficiency of 99% or more is attached to side of the box. In an embodiment of the invention, the HEPA filter has a viral filtration efficiency of at least 99.9%. This filter is connected to a vacuum which pulls over 250 cubic feet of air per minute (7000 liters per minute). In an embodiment of the invention, the filter is connected to a vacuum which pulls 250-500 cubic feet of air per minute (7000 liters per minute).

The opposite side of the box may have a large opening which should be initially closed with a drape. The opening is intended for access if an airway assistant is required. The front of the aerosol containment unit has two holes for the anesthesiologist's hands. The posterior end is open, allowing space for the patient's torso or abdomen.

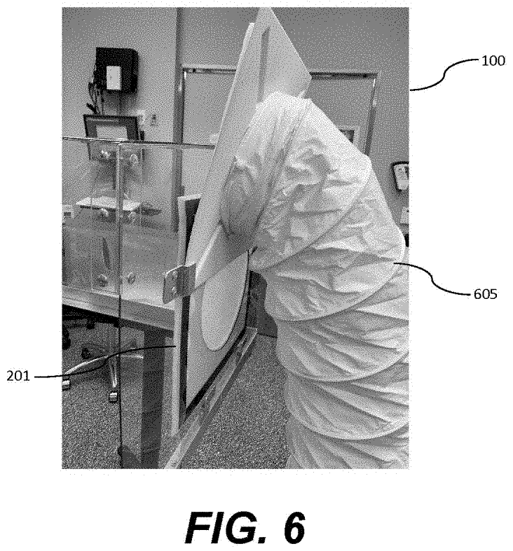

FIG. 6 illustrates how a HEPA filter HVAC unit is attached to the side panel 201 clear plastic (e.g., plexiglass) box to create the negative pressure aerosol containment unit. The flexible duct 601 of the HEPA filter HVAC unit conveys the air from the localized area of the device to create the negative pressure.

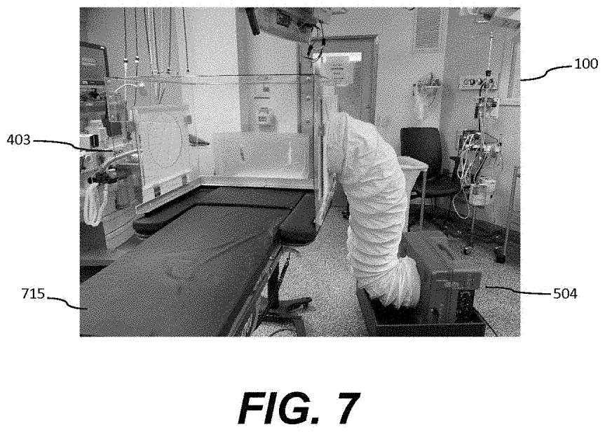

FIG. 7 illustrates the position of the aerosol containment unit on a patient bed 715. The base of the device is designed to receive the patient bed 715. This view of the invention shows HEPA filter HVAC unit connected to one side panel of the device. Side portal 403 is on the other side panel of the device. As previously discussed, the side portal 403 is designed to allow a medical or dental assistant to be able to access the box and help the primary operator working at the front of the device.

In another embodiment of the invention, the arm board of an operating room table may be positioned on their rails so that the tops of the arm boards align with the head of the bed. The device of the invention is then placed over the patient to create the negative pressure environment.

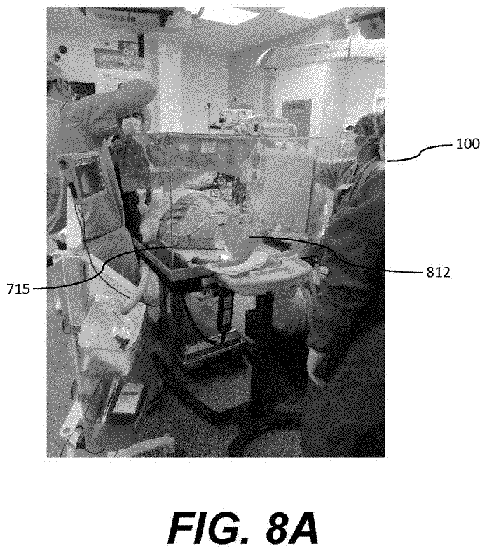

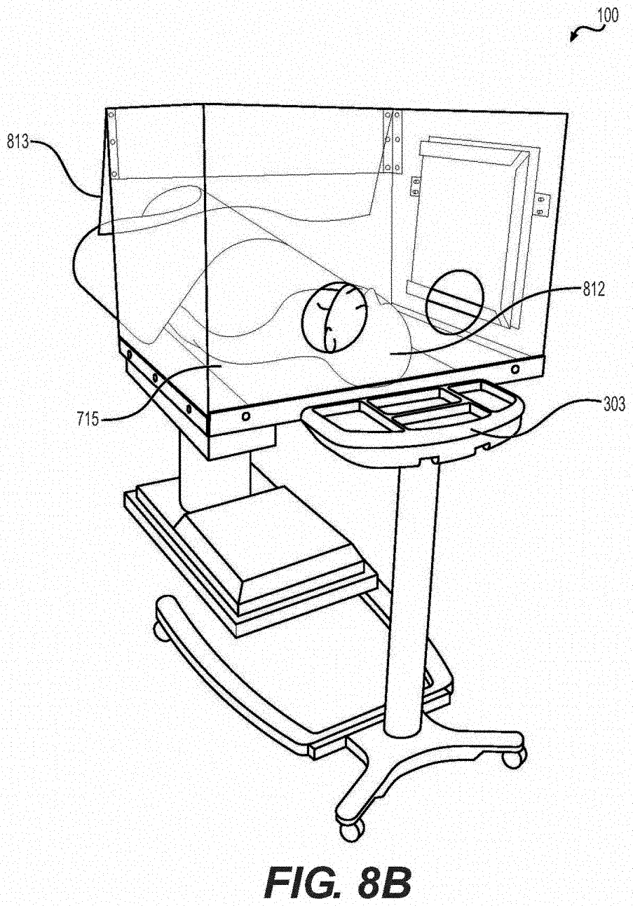

FIGS. 8A and 8B illustrate how an embodiment of the aerosol containment unit would be used on a mock patient 812 in an operating room. In this embodiment, the device is box-shaped, and the patient's head rests on the bed 715 close to the front of the box. Clinicians can be seen readying the device for use in a procedure on the patient.

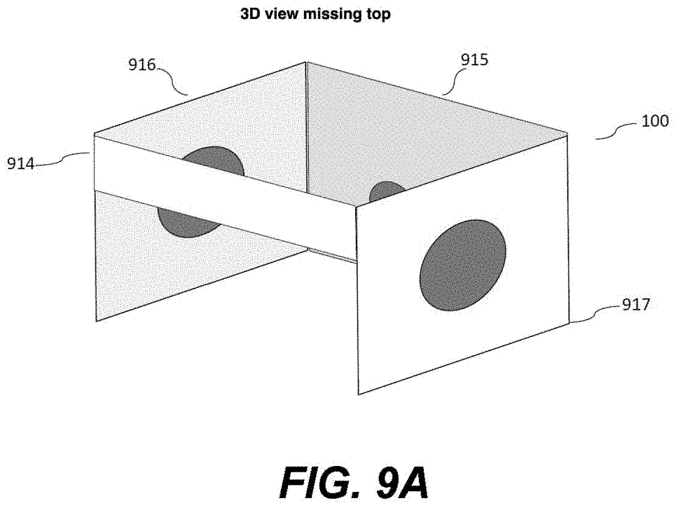

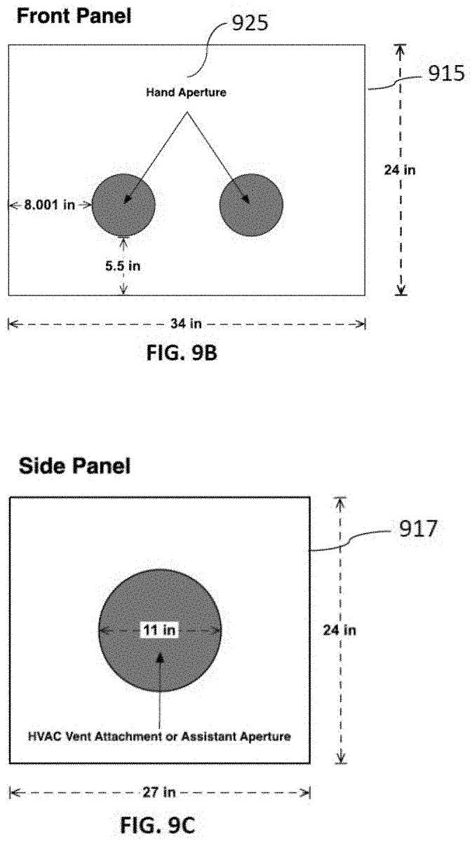

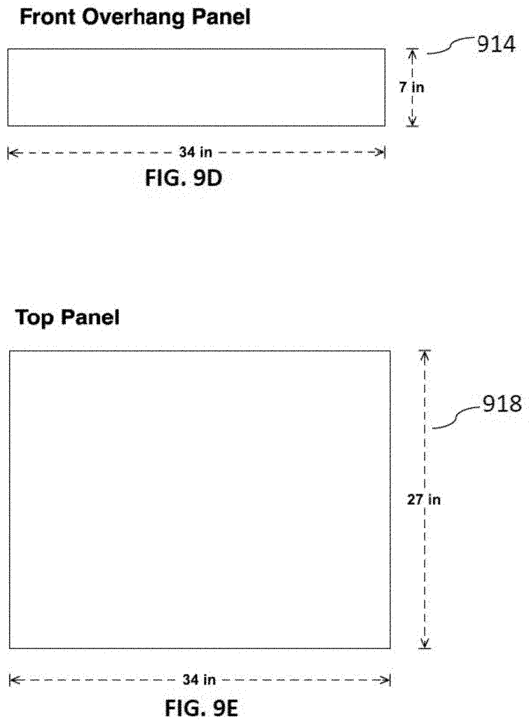

FIG. 9A is presented to show an embodiment of a device of the invention 100, the device in the form of a box and defined by a front panel 915, a partial rear or posterior panel 914, two side panels 916 and 917, a top panel (now shown in this view) and an open bottom.

Advantageously, the front panel 915, as shown in FIG. 9B, includes two hand apertures 925 that allow a clinician (e.g., a surgeon, anesthesiologist or dentist) to insert his arms and hands into the space defined by the device to perform procedures on a patient in a negative pressure environment. Such procedures include but are not limited to intubation or extubation in connection with a ventilator. While the hand apertures are not located in a set position, in this embodiment of the invention, each of the two apertures are located about 8 inches from the side of the panel and 5.5 inches from the bottom of the panel. While the dimensions of the front panel of the box can vary, in this embodiment, they are 34 inches in width by 24 inches in height.

FIG. 9C is a side view of an embodiment of the invention. This embodiment could represent either side panel in a box-shaped design of the device. In this embodiment, a circular cutout of the panel can accommodate, on the one side, an HVAC vent attachment, or, on the other side, an aperture for an assistant clinician or dental technician. In this embodiment, the side panel is 27 inches side by 24 inches in height. The dimensions of the sides of the box can vary depending on the needs of the particular device. The aperture is located in the center of the panel and is 11 inches in diameter.

FIG. 9D shows a rear overhang panel 914 on the posterior side of the invention. The overhang panel allows space for the patient's torso or abdomen as the remainder of the aerosol containment unit fits over the patient above the torso to create a negative pressure space around the upper section of the patient. Other materials may be placed on this overhang panel to minimize the open space in this posterior panel between the patient's torso and the panel. This could include, for example, a quarter or half-sheet drape. The bed height and the height of the aerosol containment device may be adjusted to optimize ergonomics for the clinicians performing a procedure on the patient.

FIG. 9E is top view of the invention showing the top pane 918, which in this embodiment is 34 inches by 27 inches.

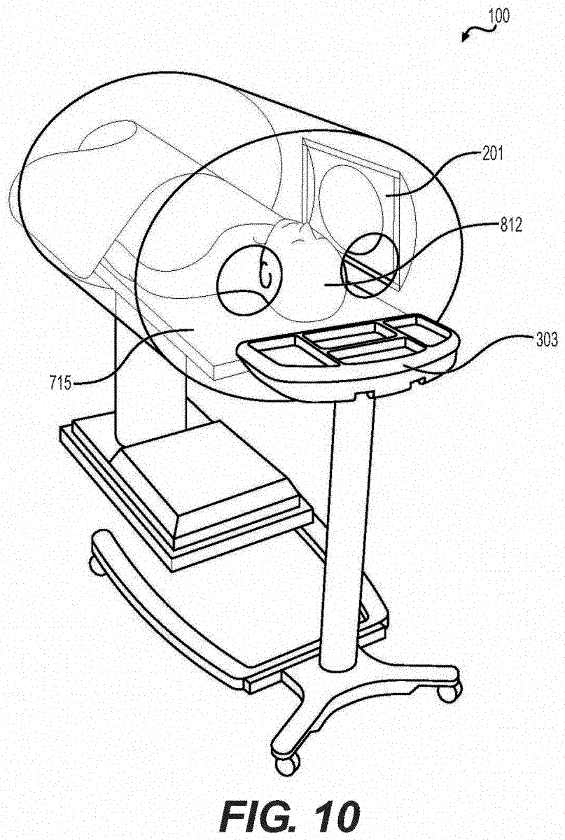

FIG. 10 shows a non-rectilinear embodiment of the invention. In this embodiment, the cross section of the unit is substantially oval in shape. As previously noted, the device, which may be mobile or portable, is not limited to a rectilinear shape and may be designed to suit the needs of the patient as well as the medical clinicians. This figure shows a mock patient 812 resisting on a patient bed 715. An optional table 303 is connected to the device in the front to allow the primary clinician to rest the tools and equipment necessary for a procedure. The unit may be configured to receive an attached HEPA filter and HVAC unit that creates a localized negative pressure area within the area in and around the device. See, for example, connection 201 for the HEPA Filter HVAC duct, which may be placed in a number of locations of a non-rectilinear device.

As the present invention may be embodied in several forms without departing from the spirit or essential characteristics thereof, it will be understood that the invention is not limited by the details of the foregoing description, unless otherwise specified, but rather should be construed broadly within its spirit and scope as defined in the appended claims, and therefore all changes and modifications that fall within the metes and bounds of the claims. Accordingly, the invention is defined by the appended claims wherein:

What is claimed is:

1. A device for use in medical and dental procedures to contain aerosols generated by a patient with a known or suspected contagious respiratory pathogen, wherein the device is transparent, has an opening at the bottom adapted to fit over the patient, and is configured to receive an attached HEPA filter and HVAC unit that creates a localized negative pressure area within the device.

2. The device of claim 1, wherein the device is in the form of a box with an open bottom.

3. The device of claim 2, wherein the box is defined by a front panel, a back panel, two side panels, a top panel and the open bottom.

4. The device of claim 3, wherein the side panels of the box are made of a clear plastic or non-porous material.

5. The device of claim 4, wherein the side panels of the box are made of plexiglass.

6. The device of claim 1, wherein the patient has a known or suspected virus.

7. The device of claim 1, wherein the virus is COVID-19.

8. The device of claim 1, wherein the device is positioned on a mobile base.

9. The device of claim 1, wherein the device is used to contain aerosols generated by a patient during intubation, extubation, bronchoscopy, tracheostomy placement, and upper endoscopy medical procedures.

10. The device of claim 1, wherein the device is used to contain aerosols generated by a patient during a dental procedure.

11. The device of claim 1, wherein the HEPA filter has a viral filtration efficiency of between 85% and 99.999995%

12. The device of claim 1, wherein the HVAC unit pulls air in a range between 250 cubic ft./minute and 600 cubic ft/minute.

13. The device of claim 3, wherein the front panel is adapted for two openings through which a clinician can engage with the patient using his hands.

14. The device of claim 13, wherein the back panel is adapted to fit over the patient's torso or abdomen.

15. The device of claim 14, wherein one side panel is configured to be fitted with the HEPA filter.

16. The device of claim 15, wherein the other side panel has a large opening configured to be covered with a drape which will allow access by an assistant clinician.
